(19) United States
(12) Patent Application Publication
(10) Pub. No.: US 2026/0209268 A1
(43) Pub. Date: Jul. 23, 2026

(54) METHODS OF REDUCING CONTAMINANTS IN PROTEIN PURIFICATION

(71) Applicant: ELI LILLY AND COMPANY, Indianapolis, IN (US)

(72) Inventor: Amy Lynn HUEBNER, Bridgewater, NJ (US)

(21) Appl. No.: 19/141,739

(22) PCT Filed: Dec. 21, 2023
(86) PCT No.: PCT/US2023/085578

§ 371 Date: Jun. 20, 2025

Related U.S. Application Data
(60) Provisional application No. 63/476,693, filed on Dec. 22, 2022.

(51) Int. Cl. C07K 1/34; C07K 1/18; C07K 1/22; C07K 1/36; C07K 14/605

(52) U.S. Cl. CPC C07K 1/34; C07K 1/18; C07K 1/22; C07K 1/36; C07K 14/605; C07K 2319/30

(57) ABSTRACT

Provided herein are improved methods for purifying an Fc-containing protein from a mixture comprising the Fc-containing protein and a contaminant (e.g., an undesired host cell protein). The methods generally comprise heating the mixture to produce a heat-treated mixture, and applying the heat-treated mixture to a depth filter to produce a filtrate comprising the Fc-containing protein.

### BACKGROUND

Production of recombinant Fc-containing proteins for therapeutic use typically involves expression of the proteins in mammalian cells and subsequent purification of these proteins from host cell contaminants. Host cell contaminants include host cell proteins (HCPs), such as proteases (e.g., cathepsin D). If these proteases are not removed, they can clip or fragment Fc-containing proteins, which reduces yield of intact protein.

Accordingly, there is a need for improved methods of purifying Fc-containing proteins to remove HCPs, such as proteases (e.g., cathepsin D).

### SUMMARY

The present disclosure provides improved methods for purifying an Fc-containing protein (e.g., dulaglutide) from a mixture comprising the Fc-containing protein and a contaminant (e.g., an HCP). The methods generally comprise heating the mixture to produce a heat-treated mixture, and applying the heat-treated mixture to a depth filter to produce a filtrate comprising the Fc-containing protein (e.g., dulaglutide). The methods disclosed herein are particularly advantageous in that they reduce the level of HCPs, including cathepsin D, and significantly increase the amount of intact Fc-containing protein recovered during purification.

In an aspect, provided herein is a method of method of purifying dulaglutide from a mixture of dulaglutide and a contaminant, the method comprising: (a) heating the mixture to produce a heat-treated mixture; and (b) applying the heat-treated mixture to a depth filter to produce a filtrate comprising dulaglutide.

In an embodiment, the mixture is heated to a temperature of 50° C. to 55° C. In an embodiment, the mixture is heated to a temperature of 51° C. to 54° C.

In an embodiment, the mixture is heated for a period of time. In an embodiment, the period of time is 120 to 180 minutes. In an embodiment, the period of time is about 120 minutes.

In an embodiment, prior to step (a), dulaglutide was purified using affinity chromatography. In an embodiment, prior to step (a), dulaglutide was purified using Protein A, Protein G, or Protein L affinity chromatography.

In an embodiment, the method further comprises purifying dulaglutide from the filtrate using ion exchange chromatography (IEX) to produce an IEX eluate. In an embodiment, the IEX is anion exchange chromatography (AEX).

In an embodiment, the depth filter is a synthetic depth filter. In an embodiment, the depth filter comprises silica filter aid and polyacrylic fiber.

In an embodiment, the depth filter comprises a filter surface area of about 0.0023 m², about 0.0135 m², about 0.027 m², about 0.054 m², about 0.11 m², about 0.33 m², about 0.55 m², about 0.77 m², or about 1.1 m².

In an embodiment, the depth filter is capable of product filtration at a flux of about 200 liters per meter squared per hour (LMH).

In an embodiment, the depth filter is capable of flush filtration at a flux of about 300 LMH.

In an embodiment, the heat-treated mixture is applied to the depth filter at a load of about 250 g/m² to about 2,000 g/m². In an embodiment, the heat-treated mixture is applied to the depth filter at a load of about 500 g/m² to about 2,000 g/m².

In an embodiment, the contaminant is one or more host cell proteins (HCPs). In an embodiment, the one or more HCPs is selected from the group consisting of one or more of a serine protease, an aspartic protease, a cysteine protease, a metalloprotease, and an aminopeptidase. In an embodiment, the aspartic protease is cathepsin D.

In an embodiment, the filtrate comprises less than about 100 ng/mg HCPs. In an embodiment, the filtrate comprises less than about 50 ng/mg HCPs, optionally less than about 10 ng/mg HCPs. In an embodiment, the filtrate comprises less than 100 μU/mL cathepsin D activity.

In an embodiment, the IEX eluate comprises less than 50 ng/mg HCPs, optionally less than 10 ng/mg HCPs. In an embodiment, the IEX eluate comprises less than 100 μU/mL cathepsin D activity, optionally less than 50 μU/mL cathepsin D activity.

In an embodiment, the concentration of the contaminant is measured by mass spectrometry or an enzyme-linked immunosorbent assay (ELISA).

In another aspect, provided herein is a composition produced by any of the methods described herein.

In another aspect, provided herein is dulaglutide produced by any of the methods described herein.

### Additional Embodiments of the Present Disclosure are Described Below

Embodiment 1. A method of purifying an Fc-containing protein from a mixture of the Fc-containing protein and a contaminant, the method comprising: (a) heating the mixture to 51° C. to 54° C. for a period of time to produce a heat-treated mixture; and (b) applying the heat-treated mixture to a depth filter to produce a filtrate comprising the Fc-containing protein.
Embodiment 2. The method of Embodiment 1, wherein the period of time is 120-180 minutes.
Embodiment 3. The method of Embodiment 1 or 2, wherein the period of time is about 120 minutes.
Embodiment 4. The method of any one of the preceding Embodiments, wherein prior to step (a), the Fc-containing protein was purified using Protein A, Protein G, or Protein L affinity chromatography.
Embodiment 5. The method of any one of the preceding Embodiments, further comprising purifying the Fc-containing protein from the filtrate using ion exchange chromatography (IEX) to produce an IEX eluate.
Embodiment 6. The method of Embodiment 5, wherein the IEX is anion exchange chromatography (AEX).
Embodiment 7. The method of any one of the preceding Embodiments, wherein the depth filter is a synthetic depth filter.
Embodiment 8. The method of any one of the preceding Embodiments, wherein the depth filter comprises silica filter aid and polyacrylic fiber.
Embodiment 9. The method of any one of the preceding Embodiments, wherein the depth filter comprises a filter surface area of about 0.0023 m², about 0.0135 m², about 0.027 m², about 0.054 m², about 0.11 m², about 0.33 m², about 0.55 m², about 0.77 m², or about 1.1 m².
Embodiment 10. The method of any one of the preceding Embodiments, wherein the depth filter is capable of product filtration at a flux of about 200 liters per meter squared per hour (LMH).
Embodiment 11. The method of any one of the preceding Embodiments, wherein the depth filter is capable of flush filtration at a flux of about 300 LMH.
Embodiment 12. The method of any one of the preceding Embodiments, wherein the heat-treated mixture is applied to the depth filter at a load of about 250 g/m² to about 2,000 g/m².
Embodiment 13. The method of any one of the preceding Embodiments, wherein the heat-treated mixture is applied to the depth filter at a load of about 500 g/m² to about 2,000 g/m².
Embodiment 14. The method of any one of the preceding Embodiments, wherein the contaminant is one or more host cell proteins (HCPs).
Embodiment 15. The method of Embodiment 14, wherein the one or more HCPs is selected from the group consisting of one or more of a serine protease, an aspartic protease, a cysteine protease, a metalloprotease, and an aminopeptidase.
Embodiment 16. The method of Embodiment 15, wherein the aspartic protease is cathepsin D.
Embodiment 17. The method of any one of the preceding Embodiments, wherein the filtrate comprises less than about 100 ng/mg HCPs.
Embodiment 18. The method of any one of the preceding Embodiments, wherein the filtrate comprises less than about 50 ng/mg HCPs, optionally less than about 10 ng/mg HCPs.
Embodiment 19. The method of any one of the preceding Embodiments, wherein the filtrate comprises less than 100 μU/mL cathepsin D activity.
Embodiment 20. The method of any one of Embodiments 5-19, wherein the IEX eluate comprises less than 50 ng/mg HCPs, optionally less than 10 ng/mg HCPs.
Embodiment 21. The method of any one of Embodiments 5-20, wherein the IEX eluate comprises less than 100 μU/mL cathepsin D activity, optionally less than 50 μU/mL cathepsin D activity.
Embodiment 22. The method of any one of Embodiments 17-21, wherein the concentration of the contaminant is measured by mass spectrometry or an enzyme-linked immunosorbent assay (ELISA).
Embodiment 23. The method of any one of the preceding Embodiments, wherein the Fc-containing protein is a glucagon-like peptide-1 (GLP-1) receptor agonist.
Embodiment 24. The method of Embodiment 23, wherein the GLP-1 receptor agonist comprises a GLP-1 analog comprising one, two, or three modifications compared to a wild type GLP-1 amino acid sequence (SEQ ID NO: 1).
Embodiment 25. The method of Embodiment 23 or 24, wherein the GLP-1 receptor agonist comprises a GLP-1 analog comprising the amino acid sequence of SEQ ID NO: 2.
Embodiment 26. The method of any one of Embodiments 23-25, wherein the GLP-1 receptor agonist comprises a peptide linker.
Embodiment 27. The method of Embodiment 26, wherein the peptide linker comprises 1 to 10 GGGGS units (SEQ ID NO: 3).
Embodiment 28. The method of any one of Embodiments 23-27, wherein the GLP-1 receptor agonist comprises: a) a GLP-1 analog comprising the amino acid sequence of SEQ ID NO: 2; b) a peptide linker comprising 1 to 10 GGGGS units (SEQ ID NO: 3); and c) a human Fc region.
Embodiment 29. The method of Embodiment 28, wherein the N-terminal glycine of the peptide linker is directly fused to the C-terminal residue of the GLP-1 analog, and the C-terminal serine of the peptide linker is directly fused to the N-terminal residue of the Fc region.
Embodiment 30. The method of any one of Embodiments 23-29, wherein the GLP-1 receptor agonist comprises the amino acid sequence of SEQ ID NO: 4.
Embodiment 31. The method of any one of Embodiments 23-30, wherein the Fc-containing protein comprises a homodimer of the amino acid sequence of SEQ ID NO: 4.
Embodiment 32. The method of Embodiment 23, wherein the Fc-containing protein is dulaglutide.
Embodiment 33. A composition produced by the method of any one of the preceding Embodiments.
Embodiment 34. Dulaglutide produced by the method of any one of the preceding Embodiments.

### BRIEF DESCRIPTION OF DRAWINGS

FIG. 1 is a flow diagram showing the various steps of three dulaglutide processing schemes as described in Example 2.

FIG. 2 is a graph showing the HCP content in ppm as assessed by ELISA detected in samples from various points of the processing strands as described in FIG. 1. “HI” represents the HCP content detected in heat inactivated samples; “DF MS” represents the HCP content detected in depth filtrate samples; and “AEX MS” represents the HCP content detected in anion exchange chromatography eluate samples.

FIG. 3 is a flow diagram showing the various steps of four dulaglutide processing schemes as described in Example 3.

### DETAILED DESCRIPTION

Provided herein are improved methods for purifying an Fc-containing protein (e.g., dulaglutide) from a mixture comprising the Fc-containing protein and a contaminant (e.g., an HCP). The methods provided herein are based in part on the finding that incorporating a downstream depth filtration step after heat inactivation, results in enhanced clearance of contaminants (e.g., HCPs). Accordingly, the methods herein generally comprise heating the mixture to produce a heat-treated mixture, and applying the heat-treated mixture to a depth filter to produce a filtrate comprising the Fc-containing protein (e.g., dulaglutide). The methods disclosed herein are particularly advantageous in that they reduce the level of HCPs, including cathepsin D, and increase the amount of intact Fc-containing protein recovered during purification.

### I. Definitions

As used herein, the term “Fc-containing protein” refers to a protein comprising an Fc region. In an embodiment, the Fc-containing protein comprises a variant Fc region comprising one or more amino acid substitutions, additions, and/or deletions relative to a naturally occurring Fc region. In an embodiment, the Fc-containing protein is not an antibody.

As used herein, the term “contaminant” refers to any material, particularly a biological macromolecule such as DNA, RNA, or a protein, other than a recombinantly produced Fc-containing protein that is present in a mixture. Contaminants include, without limitation, cellular and viral proteins or nucleic acids, or byproducts thereof, that arise in the production process of an Fc-containing protein. A contaminant also includes any host cell protein (HCP), host cell nucleic acid, or host cell fragment that results from any stage of an Fc-containing protein production process.

The terms “host cell protein,” and “HCP,” are used herein to refer to any unwanted protein that originates from a cell (e.g., a mammalian cell) used to produce an Fc-containing protein.

As used herein, the term “purifying,” “purify,” or “purification” refers to reduction in the amount of a contaminant (e.g., an HCP) in a composition comprising an Fc-containing protein. Purification may or may not result in the complete removal of contaminants from a composition. In certain embodiments, purification refers to at least a 2-fold, 3-fold, 4-fold, 5-fold, 10-fold, 15-fold, 20-fold, 25-fold, 30-fold, 35-fold, 40-fold, 45-fold, or 50-fold reduction in contaminants.

As used herein, the term “antibody” includes full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′) 2 fragments, disulfide-linked Fvs (sdFv), anti-idiotypic (anti-Id) antibodies (including, e.g., anti-anti-Id antibodies), and antigen-binding fragments of any of the above.

As used herein, the term “about,” when in reference to a value or parameter herein, includes a variability of ±5% of the value or parameter. For example, when referring to a temperature value, “about” refers to a range that includes the value 5% below the referenced value, and the value 5% above the referenced value. Thus, a temperature of about 50° C. refers to a temperature that encompasses a temperature of 47.5° C. to a temperature of 52.5° C., inclusive.

### II. Depth Filtration Methods

A challenge in the downstream processing of Fc-containing proteins (e.g., dulaglutide) is the efficient separation of the Fc-containing protein from contaminants and impurities, such as host cell proteins (HCPs). In particular, in the manufacturing process of dulaglutide, residual cathepsin D can clip the dulaglutide product and lead to a reduced yield of intact dulaglutide.

Depth filters are known to reduce process-related impurities such as HCPs. The present disclosure relates to the finding that incorporating a downstream depth filtration step after heat inactivation, results in enhanced clearance of contaminants (e.g., HCPs). Accordingly, the methods herein generally comprise heating the mixture to produce a heat-treated mixture, and applying the heat-treated mixture to a depth filter to produce a filtrate comprising the Fc-containing protein (e.g., dulaglutide). The methods disclosed herein are particularly advantageous in that they reduce the level of HCPs, including cathepsin D, and increase the amount of intact Fc-containing protein recovered during purification.

In an aspect, provided herein is a method of purifying an Fc-containing protein from a mixture comprising the Fc-containing protein and a contaminant, the method comprising heating the mixture to produce a heat-treated mixture; and applying the heat-treated mixture to a depth filter to produce a filtrate comprising the Fc-containing protein. In an embodiment, provided herein is a method of purifying dulaglutide from a mixture comprising dulaglutide and a contaminant, the method comprising heating the mixture to produce a heat-treated mixture; and applying the heat-treated mixture to a depth filter to produce a filtrate comprising dulaglutide. The step of heating the mixture to produce a heat-treated mixture generally comprises heating the mixture to a temperature, wherein the mixture is heated for a period of time.

In an embodiment, the step of heating the mixture to produce a heat-treated mixture comprises heating the mixture to a temperature of about 50° C. to about 55° C., about 50° C. to about 54° C., about 50° C. to about 53° C., about 50° C. to about 52° C., about 50° C. to about 51° C., about 51° C. to about 55° C., about 51° C. to about 54° C., about 51° C. to about 53° C., about 51° C. to about 52° C., about 52° C. to about 55° C., about 52° C. to about 54° C., about 52° C. to about 53° C., about 53° C. to about 55° C., about 53° C. to about 54° C., or about 54° C. to about 55° C. In an embodiment, the step of heating the mixture to produce a heat-treated mixture comprises heating the mixture to a temperature of about 50° C., about 51° C., about 52° C., about 53° C., about 54° C., or about 55° C.

In an embodiment, the step of heating the mixture to produce a heat-treated mixture comprises heating the mixture to a temperature, wherein the mixture is heated for about 120 to about 180 minutes, about 120 to about 170 minutes, about 120 to about 160 minutes, about 120 to about 150 minutes, about 120 to about 140 minutes, about 120 to about 130 minutes, about 130 to about 180 minutes, about 130 to about 170 minutes, about 130 to about 160 minutes, about 130 to about 150 minutes, about 130 to about 140 minutes, about 140 to about 180 minutes, about 140 to about 170 minutes, about 140 to about 160 minutes, about 140 to about 150 minutes, about 150 to about 180 minutes, about 150 to about 170 minutes, about 150 to about 160 minutes, about 160 to about 180 minutes, about 160 to about 170 minutes, or about 170 to about 180 minutes. In an embodiment, the step of heating the mixture to produce a heat-treated mixture comprises heating the mixture to a temperature, wherein the mixture is heated for about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, or about 180 minutes.

In an embodiment, the step of heating the mixture to produce a heat-treated mixture comprises heating the mixture to a temperature of about 50° C. to about 55° C.; about 50° C. to about 54° C.; about 50° C. to about 53° C.; about 50° C. to about 52° C.; about 50° C. to about 51° C.; about 51° C. to about 55° C.; about 51° C. to about 54° C.; about 51° C. to about 53° C.; about 51° C. to about 52° C.; about 52° C. to about 55° C.; about 52° C. to about 54° C.; about 52° C. to about 53° C.; about 53° C. to about 55° C.; about 53° C. to about 54° C.; about 54° C. to about 55° C.; about 50° C.; about 51° C.; about 52° C.; about 53° C.; about 54° C.; or about 55° C., wherein the mixture is heated for about 120 to about 180 minutes; about 120 to about 170 minutes; about 120 to about 160 minutes; about 120 to about 150 minutes; about 120 to about 140 minutes; about 120 to about 130 minutes; about 130 to about 180 minutes; about 130 to about 170 minutes; about 130 to about 160 minutes; about 130 to about 150 minutes; about 130 to about 140 minutes; about 140 to about 180 minutes; about 140 to about 170 minutes; about 140 to about 160 minutes; about 140 to about 150 minutes; about 150 to about 180 minutes; about 150 to about 170 minutes; about 150 to about 160 minutes; about 160 to about 180 minutes; about 160 to about 170 minutes; about 170 to about 180 minutes; about 120 minutes; about 125 minutes; about 130 minutes; about 135 minutes; about 140 minutes; about 145 minutes; about 150 minutes; about 155 minutes; about 160 minutes; about 165 minutes; about 170 minutes; about 175 minutes; or about 180 minutes.

In an embodiment, the heat-treated mixture is applied to the depth filter at a load of about 250 g/m² to about 2,000 g/m², about 500 g/m² to about 2,000 g/m², about 250 g/m², about 300 g/m², about 350 g/m², about 400 g/m², about 450 g/m², about 500 g/m², about 550 g/m², about 600 g/m², about 650 g/m², about 700 g/m², about 750 g/m², about 800 g/m², about 850 g/m², about 900 g/m², about 950 g/m², about 1,000 g/m², about 1,050 g/m², about 1,100 g/m², about 1,150 g/m², about 1,200 g/m², about 1,250 g/m², about 1,300 g/m², about 1,350 g/m², about 1,400 g/m², about 1,450 g/m², about 1,500 g/m², about 1,550 g/m², about 1,600 g/m², about 1,650 g/m², about 1,700 g/m², about 1,750 g/m², about 1,800 g/m², about 1,850 g/m², about 1,900 g/m², about 1,950 g/m², or about 2,000 g/m².

In an embodiment, the method further comprises purifying the Fc-containing protein from the filtrate using one or more chromatography purification steps. In an embodiment, the method further comprises purifying dulaglutide from the filtrate using one or more chromatography purification steps. Chromatography purification typically utilizes a solid phase chromatography medium to separate a molecule of interest from other molecules present in a mixture. In general, chromatography purification comprises separating the molecule of interest (e.g., Fc-containing protein, dulaglutide) from other molecules as a result of differences in rates at which the individual molecules of the mixture migrate through a stationary solid phase under the influence of a moving phase, or in bind and elute processes. Examples of various types of chromatography media include, without limitation, ion exchange resins (e.g., anion exchange resins), affinity resins, and multimodal (mixed mode) resins (e.g., resins that have been functionalized with ligands capable of multiple modes of interaction such as ion exchange, hydroxyapatite, affinity, size exclusion, and hydrophobic interactions). Additional chromatography purification methods include those that utilize, for example, without limitation, ion exchange membranes, hydrophobic interaction resins, ion exchange monoliths, size exclusion resins, and hydroxyapatite columns. In an embodiment, the method further comprises purifying the Fc-containing protein from the filtrate using ion exchange chromatography (IEX) to produce an IEX eluate. In an embodiment, the method further comprises purifying dulaglutide from the filtrate using ion exchange chromatography (IEX) to produce an IEX eluate. In an embodiment, the IEX is anion exchange (AEX) chromatography, and an AEX eluate is produced.

### Depth Filters

Provided herein are improved methods for purifying an Fc-containing protein (e.g., dulaglutide) from a mixture comprising the Fc-containing protein and a contaminant (e.g., an HCP). The methods generally comprise heating the mixture to produce a heat-treated mixture, and applying the heat-treated mixture to a depth filter to produce a filtrate comprising the Fc-containing protein (e.g., dulaglutide). The methods disclosed herein purify the Fc-containing protein (e.g., dulaglutide) from the contaminant (e.g., an HCP). The contaminant can be any material present at any stage of a method disclosed herein that is not the desired Fc-containing protein. Contaminants include, without limitation, viral and cellular proteins or nucleic acids, or byproducts thereof, that arise in the production process of the Fc-containing protein. Contaminants also include any undesired byproducts of the Fc-containing protein (e.g., fragments of the Fc-containing protein).

Depth filters generally achieve filtration within the depth of the filter material. One class of such filters is those that comprise a random matrix of fibers bonded (or otherwise fixed), to form a complex maze of flow channels. Particle separation in these filters generally results from entrapment by, or adsorption to, the fiber matrix. Depth filters retain particles and other impurities (e.g., HCPs) throughout the filter material allowing, in some cases, for retention of particles both larger and smaller than the pore size. Particle and impurity retention is thought to involve size exclusion and adsorption through hydrophobic, ionic and other interactions. Depth filters may contain multiple layers of depth filter material, layered in series. Employing such multiple layer depth filters can ensure that more of the mixture efficiently contacts the depth filter material, enabling a better adsorption profile for the impurities.

An example of a depth filter comprises cellulose fibers, a filter aid such as diatomaceous earth (DE), and a positively charged resin binder. Another example of a depth filter comprises silica and polyacrylic fiber. In an embodiment, the depth filter comprises synthetic material, non-synthetic material, or a combination thereof. In an embodiment, the depth filter comprises a substrate comprising one or more of a diatomaceous earth composition, a silica composition, a cellulose fiber, a polymeric fiber, a cohesive resin, and an ash composition.

In an embodiment, the depth filter comprises cellulose fibers, diatomaceous earth, and perlite. In an embodiment, the depth filter comprises two layers, wherein each layer comprises a cellulose filter matrix, and wherein the cellulose filter matrix is impregnated with a filter aid comprising one or more of diatomaceous earth or perlite. In an embodiment, the depth filter comprises two layers, wherein each layer comprises a cellulose filter matrix, wherein the cellulose filter matrix is impregnated with a filter aid comprising one or more of diatomaceous earth or perlite, and wherein each layer further comprises a resin binder. In an embodiment, the depth filter comprises cellulose fibers (as matrix) and charged surface groups (ionic charge modifications). In an embodiment, the depth filter comprises cellulose fibers (as matrix) and a cationic charge modifier that is chemically bound to the matrix components.

In an embodiment, the depth filter is a depth filter comprising synthetic material and does not comprise diatomaceous earth and/or perlite. In an embodiment, the depth filter does not contain diatomaceous earth. In an embodiment, the depth filter does not contain cellulose.

In an embodiment, the depth filter is a synthetic depth filter. In an embodiment, the depth filter consists of, or consists essentially of, synthetic materials, such as, e.g., silica, polyacrylic, and nylon. In an embodiment, the depth filter comprises silica (e.g., a silica filter aid) and polyacrylic fiber. In an embodiment, the depth filter comprises a silica filter aid, and/or polyacrylic fiber. In an embodiment, the depth filter comprises a silica filter aid, and polyacrylic fiber. In an embodiment, the depth filter comprises a silica filter aid, polyacrylic fiber, and/or non-woven material. In an embodiment, the depth filter comprises silica and polyacrylic fiber as non-woven material. In an embodiment, the depth filter comprises two layers of filter media, wherein a first layer comprises a silica, such as a silica filter aid, and a second layer comprises a polyacrylic fiber, such as a polyacrylic fiber pulp. In an embodiment, the silica filter aid is a precipitated silica filter aid. In an embodiment, the filter aid is an aspect of the filter, such as a layer, that aids with performing the filter function. In some embodiments, the silica filter aid is a silica gel filter aid.

In an embodiment, the depth filter is selected from the group consisting of an X0SP depth filter (Millistak+® HC Pro X0SP), a PDD1 depth filter (Pall/3M PDD1 SUPRAcap™-50 (SC050PDD1)), or a VR02 depth filter (Zeta Plus™ Biocap VR02). In an embodiment, the depth filter is an X0SP depth filter.

In an embodiment, the depth filter comprises a filter surface area from about 0.002 m² to about 1.5 m², e.g., a filter surface area of about 0.0023 m² or greater, about 0.0135 m² or greater, about 0.027 m² or greater, about 0.054 m² or greater, about 0.11 m² or greater, about 0.33 m² or greater, about 0.55 m² or greater, about 0.77 m² or greater, or about 1.1 m² or greater. In an embodiment, the depth filter comprises a filter surface area of from about 0.002 m² to about 1.5 m², such as at least about 0.0022 m², at least about 0.0023 m², at least about 0.0025 m², at least about 0.11 m², at least about 0.55 m², or at least about 1.1 m² or greater.

In an embodiment, the depth filter comprises an average pore size of about 0.1 μm to about 150 μm, for example, about 0.1 μm, about 1 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, or about 150 μm.

In an embodiment, the depth filtration is carried out at about 10 L to about 1000 L, about 20 L to about 800 L, about 30 L to about 600 L, about 40 L to about 440 L, about 50 L to about 200 L, about 10 L, about 20 L, about 30 L, about 40 L, about 50 L, about 100 L, about 150 L, about 200 L, about 250 L, about 300 L, about 350 L, about 400 L, about 450 L, about 500 L, about 550 L, about 600 L, about 650 L, about 700 L, about 750 L, about 800 L, about 850 L, about 900 L, about 950 L, about 1000 L or more of the heat-treated mixture per m² of depth filter surface area.

In an embodiment, the depth filter is capable of product filtration at a flux of about 100 liters per meter squared per hour (LMH) to about 300 LMH. In an embodiment, the depth filter is capable of product filtration at a flux of about 100 LMH, about 120 LMH, about 140 LMH, about 160 LMH, about 180 LMH, about 200 LMH, about 220 LMH, about 240 LMH, about 260 LMH, about 280 LMH, or about 300 LMH. In an embodiment, the depth filter is capable of product filtration at a flux of about 200 LMH.

In an embodiment, the depth filter is capable of flush filtration at a volume of about 50 L/m² to about 100 L/m². In an embodiment, the depth filter is capable of flush filtration at a volume of about 50 L/m², about 55 L/m², about 60 L/m², about 65 L/m², about 70 L/m², about 75 L/m², about 80 L/m², about 85 L/m², about 90 L/m², about 95 L/m², or about 100 L/m².

In an embodiment, the depth filter is capable of flush filtration at a flux of about 300 LMH to about 600 LMH. In an embodiment, the depth filter is capable of flush filtration at a flux of about 300 LMH, about 320 LMH, about 340 LMH, about 360 LMH, about 380 LMH, about 400 LMH, about 420 LMH, about 440 LMH, about 460 LMH, about 480 LMH, about 500 LMH, about 520 LMH, about 540 LMH, about 560 LMH, about 580 LMH, or about 600 LMH.

In an embodiment, the depth filter is capable of product filtration at a flux of about 100 LMH to about 300 LMH, and flush filtration at a flux of about 300 LMH. In an embodiment, the depth filter is capable of product filtration at a flux of about 100 LMH to about 300 LMH, and flush filtration at a flux of about 320 LMH. In an embodiment, the depth filter is capable of product filtration at a flux of about 100 LMH to about 300 LMH, and flush filtration at a flux of about 340 LMH. In an embodiment, the depth filter is capable of product filtration at a flux of about 100 LMH to about 300 LMH, and flush filtration at a flux of about 360 LMH. In an embodiment, the depth filter is capable of product filtration at a flux of about 100 LMH to about 300 LMH, and flush filtration at a flux of about 380 LMH. In an embodiment, the depth filter is capable of product filtration at a flux of about 100 LMH to about 300 LMH, and flush filtration at a flux of about 400 LMH. In an embodiment, the depth filter is capable of product filtration at a flux of about 100 LMH to about 300 LMH, and flush filtration at a flux of about 420 LMH. In an embodiment, the depth filter is capable of product filtration at a flux of about 100 LMH to about 300 LMH, and flush filtration at a flux of about 440 LMH. In an embodiment, the depth filter is capable of product filtration at a flux of about 100 LMH to about 300 LMH, and flush filtration at a flux of about 460 LMH. In an embodiment, the depth filter is capable of product filtration at a flux of about 100 LMH to about 300 LMH, and flush filtration at a flux of about 480 LMH. In an embodiment, the depth filter is capable of product filtration at a flux of about 100 LMH to about 300 LMH, and flush filtration at a flux of about 500 LMH. In an embodiment, the depth filter is capable of product filtration at a flux of about 100 LMH to about 300 LMH, and flush filtration at a flux of about 520 LMH. In an embodiment, the depth filter is capable of product filtration at a flux of about 100 LMH to about 300 LMH, and flush filtration at a flux of about 540 LMH. In an embodiment, the depth filter is capable of product filtration at a flux of about 100 LMH to about 300 LMH, and flush filtration at a flux of about 560 LMH. In an embodiment, the depth filter is capable of product filtration at a flux of about 100 LMH to about 300 LMH, and flush filtration at a flux of about 580 LMH. In an embodiment, the depth filter is capable of product filtration at a flux of about 100 LMH to about 300 LMH, and flush filtration at a flux of about 600 LMH.

In an embodiment, the depth filter is capable of product filtration at a flux of about 100 LMH and flush filtration at a flux of from about 300 LMH to about 600 LMH. In an embodiment, the depth filter is capable of product filtration at a flux of about 120 LMH and flush filtration at a flux of from about 300 LMH to about 600 LMH. In an embodiment, the depth filter is capable of product filtration at a flux of about 140 LMH and flush filtration at a flux of from about 300 LMH to about 600 LMH. In an embodiment, the depth filter is capable of product filtration at a flux of about 160 LMH and flush filtration at a flux of from about 300 LMH to about 600 LMH. In an embodiment, the depth filter is capable of product filtration at a flux of about 180 LMH and flush filtration at a flux of from about 300 LMH to about 600 LMH. In an embodiment, the depth filter is capable of product filtration at a flux of about 200 LMH and flush filtration at a flux of from about 300 LMH to about 600 LMH. In an embodiment, the depth filter is capable of product filtration at a flux of about 220 LMH and flush filtration at a flux of from about 300 LMH to about 600 LMH. In an embodiment, the depth filter is capable of product filtration at a flux of about 240 LMH and flush filtration at a flux of from about 300 LMH to about 600 LMH. In an embodiment, the depth filter is capable of product filtration at a flux of about 260 LMH and flush filtration at a flux of from about 300 LMH to about 600 LMH. In an embodiment, the depth filter is capable of product filtration at a flux of about 280 LMH and flush filtration at a flux of from about 300 LMH to about 600 LMH. In an embodiment, the depth filter is capable of product filtration at a flux of about 300 LMH and flush filtration at a flux of from about 300 LMH to about 600 LMH.

### Mixtures

Methods of the present disclosure comprise applying a heat-treated mixture to a depth filter to produce a filtrate comprising an Fc-containing protein.

As described herein, the heat-treated mixture is produced by heating a mixture comprising the Fc-containing protein (e.g., dulaglutide) and a contaminant, and in certain embodiments, the mixture is heated to a temperature of about 50° C. to about 55° C. for about 120 to about 180 minutes. The heat-treated mixture can be produced by heating any mixture comprising the Fc-containing protein (e.g., dulaglutide) and a contaminant. For example, the mixture comprising the Fc-containing protein (e.g., dulaglutide) and a contaminant may be obtained from a chromatography eluate, for example, eluted from various types of chromatography media including, without limitation, ion exchange resins (e.g., anion exchange resins), affinity resins (e.g., protein A, protein G, or protein L resins), and multimodal (mixed mode) resins (e.g., resins that have been functionalized with ligands capable of multiple modes of interaction such as ion exchange, hydroxyapatite, affinity, size exclusion, and hydrophobic interactions). In an embodiment, the mixture comprising the Fc-containing protein (e.g., dulaglutide) and a contaminant was purified using Protein A, Protein G, or Protein L affinity chromatography.

In an embodiment, the heat-treated mixture is produced by heating a mixture comprising the Fc-containing protein (e.g., dulaglutide) and a contaminant, wherein the mixture is a protein A affinity chromatography eluate. In an embodiment, the heat-treated mixture is produced by heating a mixture comprising the Fc-containing protein (e.g., dulaglutide) and a contaminant, wherein the mixture is obtained from a protein A affinity chromatography eluate. In some embodiments, the mixture is subject to one or more treatments prior to heating to produce the heat-treated mixture. For example, in an embodiment, the mixture is subject to low pH viral inactivation followed by pH neutralization. In an embodiment, the mixture is subject to low pH viral inactivation, e.g., by the addition of a buffer having a pH of from about pH 2 to about pH 4, followed by pH neutralization. In an embodiment, the mixture is subject to low pH viral inactivation, e.g., by the addition of a buffer having a pH of about pH 2, about pH 2.1, about pH 2.2, about pH 2.3, about pH 2.4, about pH 2.5, about pH 2.6, about pH 2.7, about pH 2.8, about pH 2.9, about pH 3, about pH 3.1, about pH 3.2, about pH 3.3, about pH 3.4, about pH 3.5, about pH 3.6, about pH 3.7, about pH 3.8, about pH 3.9, or about pH 4, followed by pH neutralization.

In an embodiment, a mixture comprising the Fc-containing protein (e.g., dulaglutide) and a contaminant is contacted with an affinity chromatography media (e.g., a protein A affinity chromatography media) to produce an affinity chromatography eluate comprising the Fc-containing protein and the contaminant. The affinity chromatography eluate is then treated with low pH viral inactivation followed by pH neutralization to produce a pH adjusted eluate comprising the Fc-containing protein and the contaminant. The pH adjusted eluate is then heated to a temperature of about 50° C. to about 55° C. for about 120 to about 180 minutes to produce a heat-treated mixture comprising the Fc-containing protein and the contaminant. In an embodiment, the heat-treated mixture is obtained by contacting a mixture comprising an Fc-containing protein and a contaminant with an affinity chromatography media (e.g., a protein A affinity chromatography media) to produce an affinity chromatography eluate comprising the Fc-containing protein and the contaminant, followed by treating the affinity chromatography eluate with low pH viral inactivation followed by pH neutralization to produce a pH-adjusted eluate comprising the Fc-containing protein and the contaminant, followed by heating the pH adjusted eluate to a temperature of about 50° C. to about 55° C. for about 120 to about 180 minutes to produce the heat-treated mixture. In an embodiment, the heat-treated mixture is obtained by contacting a mixture comprising an Fc-containing protein and a contaminant with a protein A affinity chromatography media to produce a protein A affinity chromatography eluate comprising the Fc-containing protein and the contaminant, followed by treating the protein A affinity chromatography eluate with low pH viral inactivation followed by pH neutralization to produce a pH-adjusted eluate comprising the Fc-containing protein and the contaminant, followed by heating the pH adjusted eluate to a temperature of about 50° C. to about 55° C. for about 120 to about 180 minutes to produce the heat-treated mixture.

In an embodiment, the contaminant is leached Protein A, a host cell nucleic acid, a fragment of the Fc-containing protein, aggregate of the Fc-containing protein, or derivative of the of the Fc-containing protein, an endotoxin, a viral contaminant, or a cell culture media component.

In an embodiment, the contaminant is one or more host cell proteins (HCPs). In an embodiment, the contaminant is an HCP selected from the group consisting of a serine protease, an aspartic protease, a cysteine protease, a metalloprotease, and an aminopeptidase. In an embodiment, the contaminant is one or more HCP selected from the group consisting of a serine protease, an aspartic protease, a cysteine protease, a metalloprotease, and an aminopeptidase, or a combination thereof. In an embodiment, the HCP is an aspartic protease. In an embodiment, the HCP is cathepsin D.

In an embodiment, the HCP is selected from the group consisting of protein S100-A6, lysosomal acid lipase/cholesteryl ester hydrolase, C—C motif chemokine 2, phospholipid transfer protein isoform X2, sulfhydryl oxidase 1 isoform X1, farnesyl pyrophosphate synthase isoform X1, retinoid-inducible serine carboxypeptidase isoform X1, T-complex protein 1 subunit delta, 60S ribosomal protein L18 isoform X1, cytoplasmic dynein 1 heavy chain 1 isoform X1, clathrin heavy chain 1 isoform X1, metalloproteinase inhibitor 1, pigment epithelium-derived factor isoform X1, acid ceramidase isoform X1, coatomer subunit beta isoform X1, 60S ribosomal protein L10a isoform X1, cullin-associated NEDD8-dissociated protein 1, alpha-L-iduronidase isoform X1, torsin-1B-like isoform X1.

In an embodiment, the HCP is selected from the group consisting of clusterin, 78 kDa glucose-regulated protein precursor, actin cytoplasmic 1, heat shock cognate 71 kDa protein, glyceraldehyde-3-phosphate dehydrogenase, thrombospondin-1 isoform X2, protein S100-A6, elongation factor 1-alpha 1, serine protease HTRA1 isoform X1, pyruvate kinase PKM isoform X1, glycogen phosphorylase brain form isoform X1, phospholipid transfer protein isoform X2, lysosomal acid lipase/cholesteryl ester hydrolase, lipoprotein lipase isoform X2, elongation factor 2, tubulin alpha-1B chain, T-complex protein 1 subunit beta isoform X1, tubulin beta chain isoform X1, alpha-enolase isoform X2, galectin-3-binding protein, cofilin-1, MAM domain-containing protein 2 isoform X1, heat shock protein HSP 90-beta, C—C motif chemokine 2, elongation factor 1-gamma, glucosylceramidase, transketolase isoform X1, T-complex protein 1 subunit theta isoform X1, alcohol dehydrogenase class-3, bone morphogenetic protein 1 partial, glycogen phosphorylase muscle form, T-complex protein 1 subunit delta, procollagen C-endopeptidase enhancer 1, T-complex protein 1 subunit eta isoform X1, ubiquitin-like modifier-activating enzyme 1 isoform X1, matrix metalloproteinase-19, GTP-binding nuclear protein Ran, T-complex protein 1 subunit zeta, sulfhydryl oxidase 1 isoform X1, phosphoglycerate kinase 1, T-complex protein 1 subunit theta isoform X2, T-complex protein 1 subunit gamma, fatty acid synthase, peroxiredoxin-1, 14-3-3 protein epsilon isoform X1, 40S ribosomal protein SA, guanine nucleotide-binding protein subunit beta-2-like 1 isoform X1 partial, 6-phosphogluconate dehydrogenase decarboxylating isoform X1, procollagen-lysine 2-oxoglutarate 5-dioxygenase 2 isoform X1, T-complex protein 1 subunit alpha, proliferation-associated protein 2G4, T-complex protein 1 subunit epsilon, disintegrin and metalloproteinase domain-containing protein 17 precursor, out at first protein homolog isoform X1 partial, pre-mRNA-processing factor 19 isoform X1, eukaryotic initiation factor 4A-I, aldose reductase isoform X1, lactadherin isoform X2 partial, 40S ribosomal protein S3 isoform X1, basement membrane-specific heparan sulfate proteoglycan core protein isoform X1, lysosomal alpha-glucosidase isoform X1, prosaposin isoform X1, pigment epithelium-derived factor isoform X1, 60S acidic ribosomal protein P0 isoform X1, heat shock protein HSP 90-alpha, MAM domain-containing protein 2 isoform X2 partial, semaphorin-3C isoform X1, hypoxia up-regulated protein 1 precursor, alanine-tRNA ligase cytoplasmic, semaphorin-3B isoform X1, clathrin heavy chain 1 isoform X1, protein CYR61 isoform X1, 14-3-3 protein theta, retinoid-inducible serine carboxypeptidase isoform X1, D-3-phosphoglycerate dehydrogenase, coatomer subunit beta isoform X1, complement C1r subcomponent, nidogen-1 isoform X1, EMILIN-1 isoform X1, tetratricopeptide repeat protein 13, endoplasmin, BTB/POZ domain-containing protein KCTD14 isoform X1, disintegrin and metalloproteinase domain-containing protein 10 isoform X1, cytoplasmic dynein 1 heavy chain 1 isoform X1, 40S ribosomal protein S2 isoform X1, farnesyl pyrophosphate synthase isoform X1, aspartate-tRNA ligase cytoplasmic isoform X1, protein S100-A10 isoform X2, adenine phosphoribosyltransferase, glutathione S-transferase P 1, serpin H1 precursor, vitamin K-dependent protein S isoform X1, sushi repeat-containing protein SRPX isoform X1, 40S ribosomal protein S3a, 26S proteasome non-ATPase regulatory subunit 1 isoform X2, platelet-derived growth factor C, 60S ribosomal protein L18 isoform X1, 60S ribosomal protein L12 isoform X1, elongation factor 1-delta isoform X3, acid ceramidase isoform X1, 26S proteasome non-ATPase regulatory subunit 13 isoform X2, glutathione S-transferase Mu 3 isoform X1, WD repeat-containing protein 1 isoform X1, ATP-citrate synthase isoform X1, 60S acidic ribosomal protein P2 isoform X1, 6-phosphofructokinase liver type isoform X1, eukaryotic translation initiation factor 6 isoform X1, alpha-L-iduronidase isoform X1, L-lactate dehydrogenase A chain isoform X2, peroxidasin homolog isoform X2, neutral alpha-glucosidase AB isoform X1, aldose reductase-related protein 2, fructose-bisphosphate aldolase A isoform X1, CMP-N-acetylneuraminate-beta-galactosamide-alpha-2,3-sialyltransferase 1 isoform X2, vacuolar protein sorting-associated protein 35 isoform X1, serpin B6 isoform X4, 60S ribosomal protein L30 isoform X1, 40S ribosomal protein S8, follistatin-related protein 1, tRNA-splicing ligase RtcB homolog isoform X1, lactadherin isoform X1 partial, transforming protein RhoA, coatomer subunit alpha, ADP-ribosylation factor 4, cullin-associated NEDD8-dissociated protein 1, rab GDP dissociation inhibitor beta, glycine-tRNA ligase isoform X1, 26S proteasome non-ATPase regulatory subunit 12 isoform X1, sphingomyelin phosphodiesterase, coatomer subunit beta' isoform X1, 26S proteasome non-ATPase regulatory subunit 3-like, thrombospondin-3 isoform X1, importin-5 isoform X1, lysyl oxidase homolog 4 isoform X1, histone H4, plasminogen activator inhibitor 1 isoform X1, torsin-1B-like isoform X1 trifunctional purine biosynthetic protein adenosine-3, tripeptidyl-peptidase 1 isoform X1, peptidyl-prolyl cis-trans isomerase FKBP4 isoform X1, exportin-1 isoform X1, 26S protease regulatory subunit 6B isoform X1, glucose-6-phosphate 1-dehydrogenase, palmitoyl-protein thioesterase 1, macrophage metalloelastase isoform X1, 26S proteasome non-ATPase regulatory subunit 7, EH domain-containing protein 4 isoform X1 partial, eukaryotic initiation factor 4A-III, vimentin, tubulointerstitial nephritis antigen-like, septin-9 isoform X2, serine-threonine kinase receptor-associated protein, hydroxymethylglutaryl-CoA synthase cytoplasmic, 40S ribosomal protein S16 isoform X1, glutathione S-transferase Mu 6, 26S proteasome non-ATPase regulatory subunit 11 isoform X1, 60S ribosomal protein L9, 40S ribosomal protein S18 isoform X1, 26S proteasome non-ATPase regulatory subunit 6, ras-related protein Rab-1A isoform X1, 60S ribosomal protein L3, 60S ribosomal protein L28 isoform X1, galectin-1, eukaryotic translation initiation factor 3 subunit L, heterogeneous nuclear ribonucleoprotein A1 isoform X1, staphylococcal nuclease domain-containing protein 1 isoform X1, ribose-phosphate pyrophosphokinase 1 isoform X1, procollagen-lysine 2-oxoglutarate 5-dioxygenase 1, 40S ribosomal protein S12 isoform X1, eukaryotic translation initiation factor 4E isoform X1, proliferating cell nuclear antigen, triosephosphate isomerase isoform X1, coatomer subunit gamma-1 isoform X1, semaphorin-3E proteasome activator complex subunit 2, ruvB-like 2 isoform X1, adenosine kinase, translational activator GCN1 isoform X1, bifunctional purine biosynthesis protein PURH isoform X1, importin subunit beta-1, protein disulfide-isomerase A3 precursor, 26S proteasome non-ATPase regulatory subunit 2 isoform X1, sushi repeat-containing protein SRPX2 isoform X1, 60S ribosomal protein L6 isoform X1, eukaryotic translation initiation factor 3 subunit I, bifunctional glutamate/proline-tRNA ligase isoform X1, dihydropyrimidinase-related protein 2 isoform X3, carbonyl reductase 1 60S ribosomal protein L13, 40S ribosomal protein S4, complement C1s subcomponent, eukaryotic translation initiation factor 3 subunit B isoform X1, V-type proton ATPase catalytic subunit A, 60S ribosomal protein L15 isoform X1, C-1-tetrahydrofolate synthase cytoplasmic, peptidyl-prolyl cis-trans isomerase B, UDP-glucuronic acid decarboxylase 1 isoform X1, 60S ribosomal protein L19, transgelin-2, peroxidasin homolog isoform X1, 60S ribosomal protein L17, eukaryotic translation initiation factor 2 subunit 3 isoform X1, integral membrane protein 2B, 60S ribosomal protein L7 isoform X2, 40S ribosomal protein S11 isoform X1, 60S ribosomal protein L10-like, S-methyl-5′-thioadenosine phosphorylase, 60S ribosomal protein L27a isoform X1, serine/threonine-protein phosphatase PP1-alpha catalytic subunit isoform X1, prostaglandin reductase 1 isoform X1, importin-7, metalloproteinase inhibitor 1, ras GTPase-activating-like protein IQGAP1 isoform X1, 40S ribosomal protein S15a, 60S ribosomal protein L26 isoform X1, metalloproteinase inhibitor 2, inorganic pyrophosphatase, 26S protease regulatory subunit 7 isoform X1, 60S ribosomal protein L5, ATP-dependent RNA helicase A isoform X1, flavin reductase (NADPH) isoform X1, poly(rC)-binding protein 2 isoform X14, protocadherin Fat 1 isoform X3, laminin subunit beta-1 isoform X1, 45 kDa calcium-binding protein isoform X2, guanine nucleotide-binding protein G (I)/G(S)/G (T) subunit beta-2 isoform X1, nucleobindin-2 isoform X1, myosin-9 isoform X1, methylosome protein 50 isoform X1 partial, MHC class I antigen Hm1-C5 isoform X1, and 26S proteasome non-ATPase regulatory subunit 3 isoform X1.

Additional examples of HCPs include, without limitation, host cell proteins involved in host cell maintenance and growth, and protein synthesis and processing, such as, e.g., phospholipase B-like 2 (PLBL2), lipoprotein lipase (LPL), lysosomal acid lipase (LAL), lysosomal lipase, (LIPA), phospholipase A2 (PLA2), palmitoyl-protein thioesterase 1 (PPT1), phospholipase B domain containing 2 (PLBD2), and peroxiredoxin.

In an embodiment, the contaminant is from a mammalian host cell. In an embodiment, the contaminant is from Chinese Hamster Ovary (CHO) cells, baby hamster kidney (BHK) cells, murine hybridoma cells, or murine myeloma cells. The contaminant may be from any host cell suitable for the production of an Fc-containing protein, for example, without limitation, HEK293 cells, NS0 cells, CHO cells, CHO-K1 cells, and CHO DG-44 cells.

### Host Cell Protein Detection

The methods provided herein result in a significant reduction in contaminants compared to other methods known in the art.

In an embodiment, the contaminant is one or more host cell proteins (HCPs). In an embodiment, the contaminant is an HCP selected from the group consisting of a serine protease, an aspartic protease, a cysteine protease, a metalloprotease, and an aminopeptidase. In an embodiment, the contaminant is one or more HCP selected from the group consisting of a serine protease, an aspartic protease, a cysteine protease, a metalloprotease, and an aminopeptidase, or a combination thereof. In an embodiment, the HCP is an aspartic protease. In an embodiment, the HCP is cathepsin D.

In an embodiment, the HCP is selected from the group consisting of protein S100-A6, lysosomal acid lipase/cholesteryl ester hydrolase, C—C motif chemokine 2, phospholipid transfer protein isoform X2, sulfhydryl oxidase 1 isoform X1, farnesyl pyrophosphate synthase isoform X1, retinoid-inducible serine carboxypeptidase isoform X1, T-complex protein 1 subunit delta, 60S ribosomal protein L18 isoform X1, cytoplasmic dynein 1 heavy chain 1 isoform X1, clathrin heavy chain 1 isoform X1, metalloproteinase inhibitor 1, pigment epithelium-derived factor isoform X1, acid ceramidase isoform X1, coatomer subunit beta isoform X1, 60S ribosomal protein L10a isoform X1, cullin-associated NEDD8-dissociated protein 1, alpha-L-iduronidase isoform X1, torsin-1B-like isoform X1.

Additional examples of HCPs include, without limitation, host cell proteins involved in host cell maintenance and growth, and protein synthesis and processing, such as, e.g., phospholipase B-like 2 (PLBL2), lipoprotein lipase (LPL), lysosomal acid lipase (LAL), lysosomal lipase, (LIPA), phospholipase A2 (PLA2), palmitoyl-protein thioesterase 1 (PPT1), phospholipase B domain containing 2 (PLBD2), and peroxiredoxin.

Methods for determining the host cell protein (HCP) concentration are known in the art. In an embodiment, an immunoassay is used to detect the amount of HCPs in a sample. In an embodiment, the immunoassay is an enzyme-linked immunosorbent assay (ELISA). In an HCP ELISA, the primary antibody is specific to the HCPs produced from a particular host cell, e.g., CHO cells, used to generate the Fc-containing protein. In an embodiment, the ELISA is a Gyrolab® CHO-HCP Kit 1 (Cygnus Technologies, Warren, NJ) ELISA assay.

In an embodiment, the amount of HCPs in a sample is measured by mass spectrometry. In an embodiment, the mass spectrometry analysis is liquid chromatography-mass spectrometry (LC-MS). In an LC-MS assay, samples are analyzed by peptide mapping/LC-MS/MS HCP profiling via, e.g., an Ultra Performance Liquid Chromatography (UPLC) coupled to a Thermo Scientific mass spectrometer. In this analysis, the samples are subjected to digestion by trypsin, reduced/precipitated with dithiothreitol (DTT), followed by transfer and acidification of the supernatant in a HPLC vial for LC-MS/MS analysis. LC-MS/MS data can be analyzed by Proteome Discoverer against a CHO-K1 protein database. The HCP content is reported as total parts per million (ppm) of HCP per sample for total HCP content (e.g., ng of HCP per mg of product).

Methods for determining the level of cathepsin D activity in a sample are known in the art. In general, any assay that can reliably detect cathepsin D activity may be used.

It has been found that in a method of purifying an Fc-containing protein as disclosed herein, comprising applying a heat-treated mixture to a depth filter to produce a filtrate, the filtrate comprises a reduced amount of one or more HCP as compared to the amount of one or more HCP in the mixture. In an embodiment, the filtrate comprises a reduced amount of cathepsin D as compared to the amount of cathepsin D in the mixture. In an embodiment, the filtrate comprises reduced cathepsin D activity compared to the level of cathepsin D activity in the mixture.

In an embodiment, the filtrate comprises less than about 100 ng/mg HCPs. In an embodiment, the filtrate comprises less than about 90 ng/mg, less than about 80 ng/mg, less than about 70 ng/mg, less than about 60 ng/mg, less than about 50 ng/mg, less than about 40 ng/mg, less than about 30 ng/mg, less than about 20 ng/mg, or less than about 10 ng/mg HCPs.

In an embodiment, the filtrate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm HCPs.

In an embodiment, the filtrate comprises less than about 90 ng/mg, less than about 80 ng/mg, less than about 70 ng/mg, less than about 60 ng/mg, less than about 50 ng/mg, less than about 40 ng/mg, less than about 30 ng/mg, less than about 20 ng/mg, less than about 10 ng/mg, less than about 9 ng/mg, less than about 8 ng/mg, less than about 7 ng/mg, less than about 6 ng/mg, less than about 5 ng/mg, less than about 4 ng/mg, less than about 3 ng/mg, less than about 2 ng/mg, or less than about 1 ng/mg cathepsin D.

In an embodiment, the filtrate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm cathepsin D.

In an embodiment, the filtrate comprises less than 300 μU/mL cathepsin D activity. In an embodiment, the eluate comprises less than 350 μU/mL, less than 325 μU/mL, less than 300 μU/mL, less than 275 μU/mL, less than 250 μU/mL, less than 225 μU/mL, less than 200 μU/mL, less than 175 μU/mL, less than 150 μU/mL, less than 125 μU/mL, less than 100 μU/mL cathepsin D activity, or less than 50 μU/mL cathepsin D activity.

In methods disclosed herein that further include purifying the Fc-containing protein from the filtrate using ion exchange chromatography (IEX), the IEX eluate comprises a reduced amount of one or more HCP as compared to the amount of one or more HCP in the mixture. In an embodiment, the IEX eluate comprises a reduced amount of cathepsin D as compared to the amount of cathepsin D in the mixture. In an embodiment, the IEX eluate comprises a reduced level of cathepsin D activity compared to the level of cathepsin D activity in the mixture.

In an embodiment, a method of purifying an Fc-containing protein as disclosed herein, comprising applying a heat-treated mixture to a depth filter to produce a filtrate followed by purifying the Fc-containing protein from the filtrate using IEX (i.e., IEX eluate obtained from a depth filtered mixture), results in an IEX eluate that comprises a reduced amount of one or more HCP as compared to the amount of one or more HCP in an IEX eluate that is obtained by a similar method that does not utilize a depth filter (i.e., IEX eluate obtained from a non-depth filtered mixture). In an embodiment, an IEX eluate obtained from a depth filtered mixture comprises a reduced amount of one or more HCP as compared to the amount of one or more HCP in an IEX eluate obtained from a non-depth filtered mixture. In an embodiment, an IEX eluate obtained from a depth filtered heat-treated mixture comprises a reduced amount of one or more HCP as compared to the amount of one or more HCP in an IEX eluate obtained from a non-depth filtered heat-treated mixture.

In an embodiment, a method of purifying an Fc-containing protein as disclosed herein, comprising applying a heat-treated mixture to a depth filter to produce a filtrate followed by purifying the Fc-containing protein from the filtrate using IEX (i.e., IEX eluate obtained from a depth filtered mixture), results in an IEX eluate that comprises a reduced amount of cathepsin D as compared to the amount of cathepsin D in an IEX eluate that is obtained by a similar method that does not utilize a depth filter (i.e., IEX eluate obtained from a non-depth filtered mixture). In an embodiment, an IEX eluate obtained from a depth filtered mixture comprises a reduced amount of cathepsin D as compared to the amount of cathepsin D in an IEX eluate obtained from a non-depth filtered mixture. In an embodiment, an IEX eluate obtained from a depth filtered heat-treated mixture comprises a reduced amount of cathepsin D as compared to the amount of cathepsin D in an IEX eluate obtained from a non-depth filtered heat-treated mixture.

In an embodiment, a method of purifying an Fc-containing protein as disclosed herein, comprising applying a heat-treated mixture to a depth filter to produce a filtrate followed by purifying the Fc-containing protein from the filtrate using IEX (i.e., IEX eluate obtained from a depth filtered mixture), results in an IEX eluate that comprises a reduced level of cathepsin D activity compared to the level of cathepsin D activity in an IEX eluate that is obtained by a similar method that does not utilize a depth filter (i.e., IEX eluate obtained from a non-depth filtered mixture). In an embodiment, an IEX eluate obtained from a depth filtered mixture comprises a reduced level of cathepsin D activity compared to the level of cathepsin D activity in an IEX eluate obtained from a non-depth filtered mixture. In an embodiment, an IEX eluate obtained from a depth filtered heat-treated mixture comprises a reduced level of cathepsin D activity compared to the level of cathepsin D activity in an IEX eluate obtained from a non-depth filtered heat-treated mixture.

In an embodiment, the IEX eluate comprises less than about 100 ng/mg HCPs. In an embodiment, the eluate comprises less than about 90 ng/mg, less than about 80 ng/mg, less than about 70 ng/mg, less than about 60 ng/mg, less than about 50 ng/mg, less than about 40 ng/mg, less than about 30 ng/mg, less than about 20 ng/mg, or less than about 10 ng/mg HCPs.

In an embodiment, the IEX eluate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm HCPs.

In an embodiment, the IEX eluate comprises less than about 90 ng/mg, less than about 80 ng/mg, less than about 70 ng/mg, less than about 60 ng/mg, less than about 50 ng/mg, less than about 40 ng/mg, less than about 30 ng/mg, less than about 20 ng/mg, less than about 10 ng/mg, less than about 9 ng/mg, less than about 8 ng/mg, less than about 7 ng/mg, less than about 6 ng/mg, less than about 5 ng/mg, less than about 4 ng/mg, less than about 3 ng/mg, less than about 2 ng/mg, or less than about 1 ng/mg cathepsin D.

In an embodiment, the IEX eluate comprises less than about 100 ppm, less than about 90 ppm, less than about 80 ppm, less than about 70 ppm, less than about 60 ppm, less than about 50 ppm, less than about 40 ppm, less than about 30 ppm, or to less than about 20 ppm cathepsin D.

In an embodiment, the IEX eluate comprises less than 300 μU/mL cathepsin D activity. In an embodiment, the eluate comprises less than 350 μU/mL, less than 325 μU/mL, less than 300 μU/mL, less than 275 μU/mL, less than 250 μU/mL, less than 225 μU/mL, less than 200 μU/mL, less than 175 μU/mL, less than 150 μU/mL, less than 125 μU/mL, less than 100 μU/mL cathepsin D activity, or less than 50 μU/mL cathepsin D activity.

In an embodiment, the amount of HCPs is measured by mass spectrometry or ELISA. In an embodiment, the amount of cathepsin D is measured by mass spectrometry or ELISA. In an embodiment, the mass spectrometry is LC-MS.

### III. Fc-Containing Proteins

The methods provided by the present disclosure are for the purification of an Fc-containing protein from a mixture of the Fc-containing protein and one or more contaminant.

The Fc-containing protein can be produced from any host cell. In an embodiment, the Fc-containing protein was produced in a mammalian host cell. In an embodiment, the Fc-containing protein was produced in Chinese Hamster Ovary (CHO) cells, baby hamster kidney (BHK) cells, murine hybridoma cells, or murine myeloma cells. Examples of mammalian host cells include, without limitation, HEK293 cells, NS0 cells, CHO cells, CHO-K1 cells, and CHO DG-44 cells.

In an embodiment, the Fc-containing protein comprises one or more of the amino acid sequences set forth in Table 1 below.

In an embodiment, the GLP-1 receptor agonist comprises a GLP-1 analog comprising one, two, or three modifications compared to a wild type GLP-1 amino acid sequence (SEQ ID NO: 1). In an embodiment, the Fc-containing protein comprises a glucagon-like peptide 1 (GLP-1) analog comprising one or more modifications compared to a wild type GLP-1 amino acid sequence (SEQ ID NO: 1).

In an embodiment, the Fc-containing protein comprises a GLP-1 analog comprising the amino acid sequence of SEQ ID NO: 2.

In an embodiment, the Fc-containing protein comprises a peptide linker. In an embodiment, the C-terminal amino acid of the GLP-I analog portion of the Fc-containing protein is fused to the N-terminus of an Fc portion of an immunoglobulin via a peptide linker. In an embodiment, the peptide linker comprises 1 to 10 G4S units (SEQ ID NO: 3).

In an embodiment, the Fc-containing protein comprises: a GLP-1 analog comprising the amino acid sequence of SEQ ID NO: 2; a peptide linker comprising 1 to 10 G4S units (SEQ ID NO: 3); and an Fc portion of an immunoglobulin. In an embodiment, the N-terminal glycine of the peptide linker is directly fused to the C-terminal residue of the GLP-1 analog, and the C-terminal serine of the peptide linker is directly fused to the N-terminal residue of the Fc portion.

In an embodiment, the Fc-containing protein is a homodimer comprising two identical amino acid chains. In an embodiment, each amino acid chain comprises the amino acid sequence of SEQ ID NO: 4.

In an embodiment, the Fc-containing protein is dulaglutide.

In an aspect, provided herein are methods for the purification of dulaglutide from a mixture of dulaglutide and one or more HCP. In an embodiment, dulaglutide is produced in CHO cells.

Dulaglutide is a human GLP-1 receptor agonist which comprises a dimer of a GLP-1 analog fused at its C-terminus via a (G4S) 3 peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO: 4.

The two monomers are attached by disulfide bonds between the cysteine residues at positions 55 and 58 of SEQ ID NO: 4 to form the dimer. Dulaglutide's structure, function, production, and use in treating T2DM is described in more detail in U.S. Pat. No. 7,452,966 and U.S. Patent Application Publication No. US20100196405, the disclosures of which are herein incorporated by reference in their entireties. Dulaglutide agonizes the GLP-1 receptor resulting in stimulation of insulin synthesis and secretion and has been shown to provide improved glycemic control in T2DM patients.

When used herein, the term “dulaglutide” refers to any GLP-1 receptor agonist protein dimer of two monomers having the amino acid sequence of SEQ ID NO: 4, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term.

TABLE 1
Sequences of dulaglutide

| | SEQ | |
|---|---|---|
| Description | ID NO: | Amino acid sequence |
| WT GLP 1 | 1 | HAEGTFTSDVSSYLEGQAAKEFIAWLVKG |
| | | RG |
| GLP 1 | 2 | HGEGTFTSDVSSYLEEQAAKEFIAWLVKG |
| analog | | GG |
| G₄S | 3 | GGGGS |
| Dulaglutide | 4 | HGEGTFTSDVSSYLEEQAAKEFIAWLVKG |
| monomer | | GGGGGGSGGGGSGGGGSAESKYGPPCPPC |
| | | PAPEAAGGPSVFLFPPKPKDTLMISRTPE |
| | | VTCVVVDVSQEDPEVQFNWYVDGVEVHNA |
| | | KTKPREEQFNSTYRVVSVLTVLHQDWLNG |
| | | KEYKCKVSNKGLPSSIEKTISKAKGQPRE |
| | | PQVYTLPPSQEEMTKNQVSLTCLVKGFYP |
| | | SDIAVEWESNGQPENNYKTTPPVLDSDGS |
| | | FFLYSRLTVDKSRWQEGNVFSCSVMHEAL |
| | | HNHYTQKSLSLSLG |

In an embodiment, the Fc-containing protein is etanercept, alefacept, abatacept, rilonacept, romiplostim, belatacept, aflibercept, conbercept, efmoroctocog alpha, eftrenonacog alpha, asfotase alpha, or luspatercept.

In an aspect, provided herein is an Fc-containing protein produced by any one of the methods disclosed herein.

In an aspect, provided herein is dulaglutide produced by any one of the methods disclosed herein.

### EXAMPLES

The following examples are offered by way of illustration, and not by way of limitation.

### Example 1: Comparison of HCP Clearance by Depth Filtration of Process Intermediates

Depth filtration is typically performed as part of a cell culture medium clarification step in the upstream production process of proteins, to remove biomass and cell debris, including host cell protein (HCP) contaminants. To determine whether incorporating depth filtration into a downstream production process that comprises protein A chromatography, followed by a low pH viral inactivation (LpHVI) and subsequent neutralization, followed by heat inactivation (HI), followed by anion exchange chromatography (AEX) can further reduce HCPs, two different dulaglutide production process intermediates were subjected to depth filtration and the extent of HCP removal evaluated. The two dulaglutide production process intermediates were: a LpHVI intermediate and a HI intermediate. The LpHVI intermediate was produced by subjecting a column chromatography eluate to low pH viral inactivation followed by pH neutralization. The HI intermediate was produced by heating the LpHVI intermediate at 51° C. to 54° C. for about 120 minutes. These process intermediates were subjected to depth filtration under the conditions set forth in Table 2.

TABLE 2
Depth Filtration Operating Conditions

| | |
|---|---|
| Operating Parameters | Operating Conditions |

| Filter Type | Millipore ® X0SP Depth Filter | |
|---|---|---|
| Filter Surface Area | 23 | cm² |
| Target Load Ratio | 2000 | g/m² |

| | |
|---|---|
| Flush Buffer | Water |
| Flush Volume and Flow Rate | ≥50 L/m² at 11.5 mL/min |
| Product Filtration Flow Rate | 200 LMH (7.7 mL/min) |
| Intermediate (“Slip-stream”) | Every 500 g/m² |
| Sampling Load Ratio | |

Each of the LpHVI intermediate and HI intermediate were tested in duplicate. The duplicate filtrations for both the LpHVI and HI intermediate all reached the target loading of 2000 g/m². The change in pressure (ΔP) for the filtrations was <3 psi, with no appreciable increase in ΔP throughout the filtration.

After depth filtration, the level of HCP was determined by both ELISA and Liquid Chromatography Mass Spectrometry (LC-MS) of the loading sample and of various slip-stream samples taken every 500 g/m² of load.

HCP ELISA was performed using a Gyrolab® CHO-HCP Kit 1 (Cygnus Technologies), according to the manufacturer's instructions. The HCP ELISA results for the slip-stream samples of all four filters showed increasing levels of HCP as the filter loading increased (increases of about 25 ppm for the LpHVI intermediate samples and 15 ppm for the HI intermediate samples). The HCP ELISA values for the filtrate pools were approximately equivalent to an average of the slip-stream samples, suggesting a linear increase in HCP breakthrough. HCP ELISA results are shown in Table 3.

TABLE 3
HCP ELISA Results

| | | | | | Change | Decrease |
|---|---|---|---|---|---|---|
| | | Conc. | HCP | HCP | in HCP | in HCP |
| Sample Description | | (mg/mL) | (ng/ml) | (ng/mg) | (ng/mg) | (%) |
| LpHVI - loading sample | | 2.61 | 362.98 | 139 | N/A | N/A |
| LpHVI Filtrate | 500 g/m² Loading | 2.76 | 85.61 | 31 | 108 | 78% |
| (Filter 1) | 1000 g/m² Loading | 2.51 | 112.94 | 45 | 94 | 68% |
| | 1500 g/m² Loading | 2.72 | 106.91 | 39 | 100 | 72% |
| | 2000 g/m² Loading | 2.51 | 136.07 | 54 | 85 | 61% |
| | Filtrate Pool | 2.42 | 102.06 | 42 | 97 | 70% |
| LpHVI Filtrate | 500 g/m² Loading | 2.74 | 74.29 | 27 | 112 | 80% |
| (Filter 2) | 1000 g/m² Loading | 2.48 | 111.9 | 45 | 94 | 68% |
| | 1500 g/m² Loading | 2.72 | 106.49 | 39 | 100 | 72% |
| | 2000 g/m² Loading | 2.52 | 131.79 | 52 | 87 | 63% |
| | Filtrate Pool | 2.41 | 103.95 | 43 | 96 | 69% |
| HI - loading sample | | 2.49 | 268.38 | 108 | N/A | N/A |
| HI Filtrate | 500 g/m² Loading | 2.63 | 43.22 | 16 | 92 | 85% |
| (Filter 3) | 1000 g/m² Loading | 2.43 | 112.94 | 26 | 82 | 76% |
| | 1500 g/m² Loading | 2.66 | 68.91 | 26 | 82 | 76% |
| | 2000 g/m² Loading | 2.45 | 136.07 | 30 | 78 | 72% |
| | Filtrate Pool | 2.34 | 102.06 | 27 | 81 | 75% |
| HI Filtrate | 500 g/m² Loading | 2.65 | 46.83 | 18 | 90 | 84% |
| (Filter 4) | 1000 g/m² Loading | 2.43 | 111.9 | 27 | 81 | 75% |
| | 1500 g/m² Loading | 2.65 | 66.29 | 25 | 83 | 77% |
| | 2000 g/m² Loading | 2.44 | 131.79 | 32 | 76 | 70% |
| | Filtrate Pool | 2.35 | 103.95 | 25 | 83 | 77% |

As shown in Table 3, the depth filtrate of HI intermediate samples contained a lower amount of HCP as compared to the amount of HCP in the depth filtrate of LpHVI intermediate samples.

To determine whether depth filtration of HI intermediate samples affects the level of HCP that remain after downstream polishing, depth filtered and non-depth filtered HI intermediate samples were subjected to anion exchange chromatography (AEX).

TABLE 4
HCP ELISA Results

| | | | | Change | Decrease |
|---|---|---|---|---|---|
| | Conc. | HCP | HCP | in HCP | in HCP |
| Sample Description | (mg/mL) | (ng/ml) | (ng/mg) | (ng/mg) | (%) |
| AEX Eluate Run #1 | 2.56 | 39.46 | 15 | 93 | 86% |
| AEX Eluate Run #2 | 2.56 | 12.25 | 5 | 103 | 96% |

Table 4 shows that AEX eluate obtained from using depth filtered HI intermediate as load (AEX Eluate Run #2) contained a significantly lower amount of HCP as compared to the amount of HCP in the AEX eluate obtained from using non-depth filtered HI intermediate as load (AEX Eluate Run #1).

For HCP LC-MS analysis, samples were subjected to trypsin digestion, reduced/precipitated with dithiothreitol (DTT), followed by transfer and acidification of the supernatant in an HPLC vial for LC-MS/MS analysis. LC-MS data was analyzed by Proteome Discoverer software (ThermoFisher) using a CHO-K1 protein database with added control protein sequences. HCP LC-MS results are shown in Table 5.

TABLE 5
HCP LC-MS Results

| | LpHVI | | | HI | | | AEX Eluate | |
|---|---|---|---|---|---|---|---|---|
| | Load | Filtrate 1 | Filtrate 2 | Load | Filtrate 1 | Filtrate 2 | Run #1 | Run #2 |
| Total HCP (ppm) | 8868 | 5888 | 4763 | 9561 | 4562 | 4474 | 1109 | 630 |
| Decrease in HCP (%) | N/A | 34% | 46% | N/A | 52% | 53% | 88% | 93% |
| Total HCP species | 230 | 196 | 185 | 229 | 188 | 189 | 16 | 10 |
| Decrease in HCP species (%) | N/A | 15% | 20% | N/A | 18% | 17% | 93% | 96% |

As shown in Table 5, the depth filtrations resulted in an approximate 40% reduction in HCP for the LpHVI intermediate samples and 52% for the HI intermediate samples. Depth filtration of LpHVI intermediates and depth filtration of HI intermediates both resulted in reduction in the number of different HCP species detected.

Table 5 also shows the results of HCP LC-MS analysis of the AEX Eluate Run #1 and AEX Eluate Run #2 samples described above. Specifically, AEX Eluate Run #2 contained less total HCP and less HCP species as compared to the amount of HCP in AEX Eluate Run #1.

The foregoing results demonstrate that: (1) the use of depth filtration as a downstream purification step in a dulaglutide production process reduces the level of HCP, and (2) depth filtration of heat inactivated dulaglutide process intermediates reduces the level HCP relative to depth filtration of corresponding dulaglutide process intermediates that are not heat inactivated.

### Example 2: Comparison of HCP Clearance Between Process Schemes with or without Downstream Depth Filtration

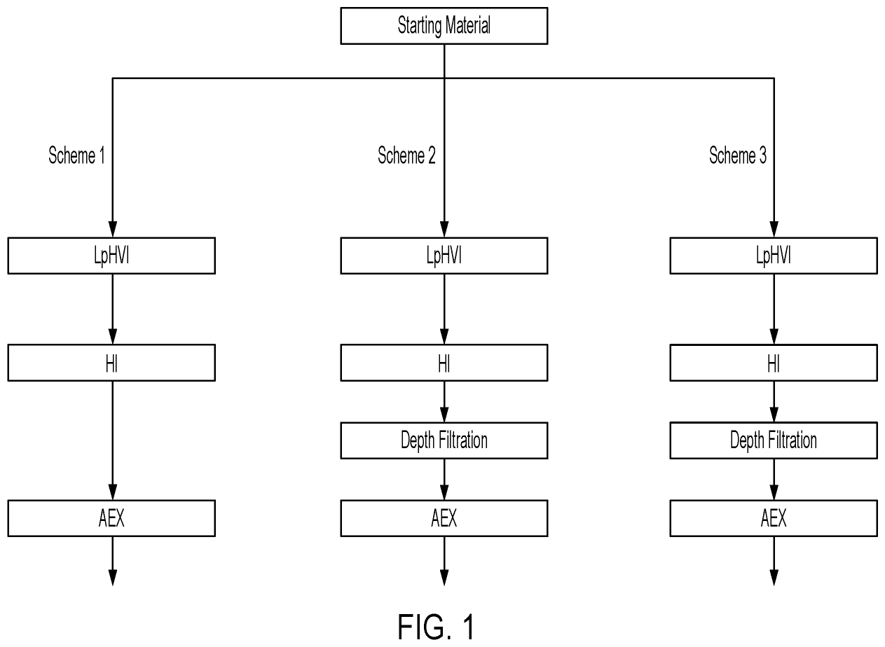

To confirm the effectiveness in reducing HCP of incorporating depth filtration into the downstream production process, the different dulaglutide purification schemes set forth in FIG. 1 were assessed. The starting material was a protein A chromatography eluate. The starting material for Scheme 1 and Scheme 2 was produced using identical chromatography methods. The only difference between Scheme 2 and Scheme 3 was in the protein A chromatography wash conditions used to obtain the starting material. The LpHVI, depth filtrationX steps, and anion exchange chromatography (“AEX”) steps were identical across the different schemes. The heat inactivation step was 51.5° C. to 52.2° C. for 120 minutes for Scheme 1; 51.0° C. to 51.5° C. for 127 minutes for Scheme 2; and 51.5° C. to 51.6° C. for 125 minutes for Scheme 3. Millipore® X0SP depth filters were used in the depth filtration steps.

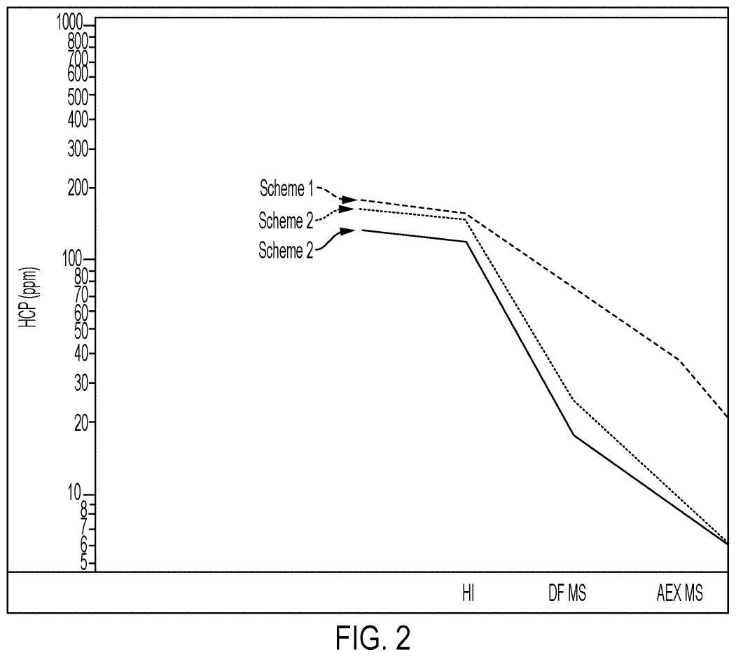

Various criteria were analytically tested at various points of the processing schemes, including level of dulaglutide aggregation, dulaglutide purity, level of dulaglutide cleavage products, and level of HCP present. All criteria were found to be similar across the different schemes, with the exception of HCP clearance, which was found to be significantly higher in Scheme 2 and Scheme 3 than Scheme 1 at all steps after depth filtration, as measured by ELISA (FIG. 2).

The foregoing results further demonstrate that the use of depth filtration as a downstream purification step in a dulaglutide production process reduces the level of HCP.

### Example 3: Comparison of HCP Clearance Between Process Schemes with or without Heat Inactivation or Downstream Depth Filtration

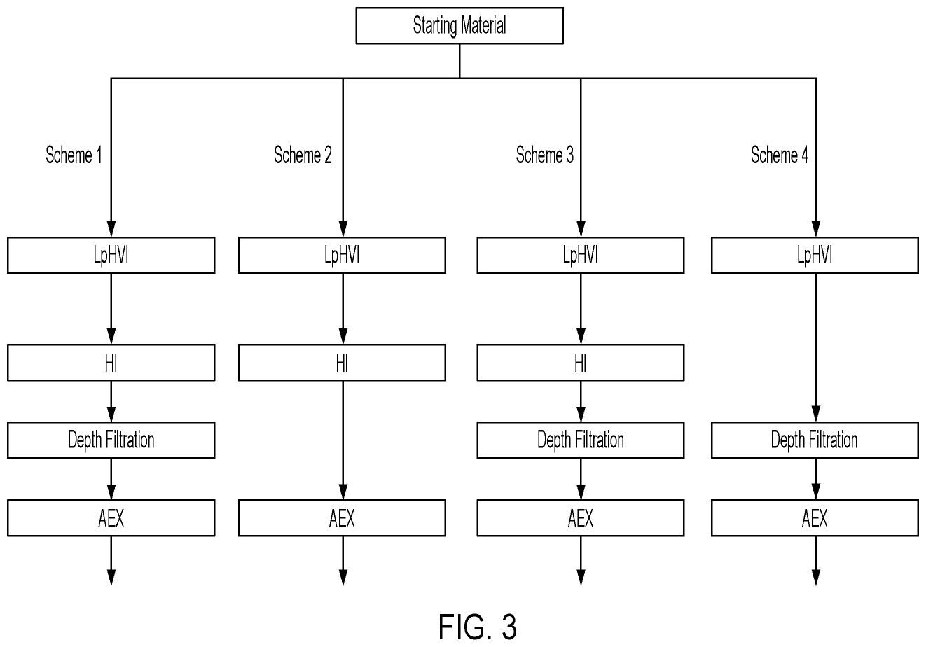

To further confirm the effectiveness in reducing HCP of incorporating depth filtration into the downstream production process, the different dulaglutide purification schemes set forth in FIG. 3 were assessed. The starting material was protein A chromatography eluate. The starting material for Schemes 2, 3 and 4 was produced using identical chromatography methods. The only difference between Scheme 1 and Scheme 3 was in the protein A chromatography wash conditions used to obtain the starting material. The LpHVI, HI, depth filtration steps, and AEX steps were identical across the different schemes. Millipore® X0SP depth filters were used in the depth filtration steps.

The level of HCP was measured in various process intermediates by ELISA and LC-MS using the methods described in Example 1 herein. A cathepsin D activity assay was also performed on the various process intermediates. Monitoring and reducing cathepsin D activity is important in dulaglutide purification processes because residual cathepsin D can proteolytically clip dulaglutide. Results from the HCP testing and cathepsin D activity assays are shown in Table 6.

TABLE 6
HCP and Cathepsin D Activity Measured
in Process Run Intermediates

| | | | Residual HCP | Cathepsin D |
|---|---|---|---|---|
| | | Residual HCP | (LC-MS; | Activity Assay |
| Sample | Run | (ELISA; ng/mg) | ppm/Dula) | (μU/mL) |
| LpHVI | 1 | 122 | 4057 | 686 |
| | 2 | 70 | 2912 | 229 |
| | 3 | 67 | 2702 | 99 |
| | 4 | 61 | 2092 | 126 |
| HI | 1 | 84 | 4211 | 50 |
| | 2 | 64 | 2837 | 28 |
| | 3 | 68 | 2725 | 45 |
| | 4 | No heat inactivation step | | |
| Depth | 1 | 16 | 1112 | 58 |
| Filtration | 2 | No depth filtration step | | |
| | 3 | 17 | 768 | 55 |
| | 4 | 20 | 966 | 264 |

As shown in Table 6, comparing the residual HCP level in depth filtrate to the HCP level in LpHVI intermediate samples, the percent reduction in residual HCP in depth filtrate from Scheme 1 and Scheme 3 (ELISA: 86.9%, LC-MS: 72.6%; and ELISA: 74.6%, LC-MS: 71.6%, respectively) was higher than the percent reduction in residual HCP in the depth filtrate from Scheme 4 (ELISA: 67.2%, LC-MS: 53.8%). Further, cathepsin D activity was lower in depth filtrates from Scheme 1 and Scheme 3, as compared to the depth filtrate from Scheme 4.

The foregoing results further confirm the findings described in Example 1 and Example 2.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Other embodiments are within the following claims.

1. A method of purifying dulaglutide from a mixture of dulaglutide and a contaminant, the method comprising:
(a) heating the mixture to produce a heat-treated mixture; and
(b) applying the heat-treated mixture to a depth filter to produce a filtrate comprising dulaglutide.

2. The method of claim 1, wherein the mixture is heated to a temperature of 50° C. to 55° C.

3. The method of claim 1, wherein the mixture is heated to a temperature of 51° C. to 54° C.

4. The method of claim 1, wherein the mixture is heated for a period of time.

5. The method of claim 4, wherein the period of time is 120 to 180 minutes.

6. The method of claim 4, wherein the period of time is about 120 minutes.

7. The method of claim 1, wherein prior to step (a), the dulaglutide was purified using affinity chromatography.

8. The method of claim 1, wherein prior to step (a), the dulaglutide was purified using Protein A, Protein G, or Protein L affinity chromatography.

9. The method of claim 1, further comprising purifying the dulaglutide from the filtrate using ion exchange chromatography (IEX) to produce an IEX eluate.

10. The method of claim 5, wherein the IEX is anion exchange chromatography (AEX).

11. The method of claim 1, wherein the depth filter is a synthetic depth filter.

12. The method of claim 11, wherein the depth filter comprises silica filter aid and polyacrylic fiber.

13. The method of claim 12, wherein the depth filter comprises a filter surface area of about 0.0023 m², about 0.0135 m², about 0.027 m², about 0.054 m², about 0.11 m², about 0.33 m², about 0.55 m², about 0.77 m², or about 1.1 m².

14. The method of claim 13, wherein the depth filter is capable of product filtration at a flux of about 200 liters per meter squared per hour (LMH).

15. The method of claim 14, wherein the depth filter is capable of flush filtration at a flux of about 300 LMH.

16. The method of claim 1, wherein the heat-treated mixture is applied to the depth filter at a load of about 250 g/m² to about 2,000 g/m².

17. The method of claim 1, wherein the heat-treated mixture is applied to the depth filter at a load of about 500 g/m² to about 2,000 g/m².

18. The method of claim 1, wherein the contaminant is one or more host cell proteins (HCPs).

19. The method of claim 18, wherein the one or more HCPs is selected from the group consisting of one or more of a serine protease, an aspartic protease, a cysteine protease, a metalloprotease, and an aminopeptidase.

20. The method of claim 19, wherein the one or more HCPs is cathepsin D.

21. The method of claim 20, wherein the filtrate comprises less than about 100 ng/mg HCPs.

22. The method of claim 20, wherein the filtrate comprises less than about 50 ng/mg HCPs.

23. The method of claim 20, wherein the filtrate comprises less than 100 μU/mL cathepsin D activity.

24. The method of claim 9, wherein the IEX eluate comprises less than 50 ng/mg HCPs.

25. The method of claim 9, wherein the IEX eluate comprises less than 100 μU/mL cathepsin D activity.

26. The method of claim 18, wherein the concentration of the HCPs is measured by mass spectrometry or an enzyme-linked immunosorbent assay (ELISA).

27. A composition produced by the method of claim 1.

28. Dulaglutide produced by the method of claim 1.

29. A composition comprising dulaglutide produced by the method of claim 1.
